(19) United States
(12) Patent Application Publication
(10) Pub. No.: US 2010/0292723 A1
(43) Pub. Date: Nov. 18, 2010

(54) DISK NEEDLE ROLLER

(76) Inventor: Myeong In Lee, Incheon (KR)

Correspondence Address: SHERR & VAUGHN, PLLC, 620 HERNDON PARKWAY, SUITE 320, HERNDON, VA, 20170, US

(21) Appl. No.: 12/746,554

(22) PCT Filed: Jun. 16, 2008
(86) PCT No.: PCT/KR2008/003384

§ 371 Date: Jun. 7, 2010

(30) Foreign Application Priority Data
Dec. 7, 2007 (KR) 10-2007-0127110
Dec. 7, 2007 (KR) 10-2007-0127111
Jun. 10, 2008 (KR) 10-2008-0054146

(51) Int. Cl. A61B 17/34

(52) U.S. Cl. 606/180

(57) ABSTRACT

The present invention relates to a disk needle roller (100) for forming micro-holes in a surface of skin by using micro-needles (220) to stimulate and assist regeneration of the skin. The disk needle roller (100) includes a handle (110) having one pair of supporting bars (120) extended therefrom, and a roller (200) including a plurality of circular spacers (232, 233) placed on a rotation shaft (130) having opposite ends supported on the supporting bars (120) of the handle (110), a plurality of circular disks (210) each between adjacent circular spacers (232, 233) so that the circular disks (210) are rotatable around the rotation shaft (130) independent from one another, a plurality of needles (220) projected from an outside circumference of each of the disks (210) in a radial direction, each with a tip formed sharply for forming micro-holes in skin of a human body.

### TECHNICAL FIELD

The present invention relates to a disk needle roller for forming micro-holes in a surface of skin by using micro-needles to stimulate and assist regeneration of the skin.

### BACKGROUND ART

A medical practice in which micro-needles are used to form a plurality of microholes in skin to assist regeneration of the skin has been recognized as an effective treatment enough to be used widely in dermatology treatments and cosmetic fields. In such a micro-needle practice, a micro-needle roller is used.

Referring to FIG. 1, the micro-needle roller is provided with a handle 10 and a roller 30. The handle 10 is long for easy holding by a user or a practitioner, and has one pair of supporting bars extended from one end for rotatably securing the roller 30 thereto. Between the one pair of supporting bars 11, the roller 30 is placed, and a rotation shaft 20 is placed to pass through both the supporting bars 11 and the roller 30, thereby rotatably securing the roller 30 to the supporting bars 11.

Referring to FIG. 2, the roller 30 has a cylindrical shape of stack of circular disks 31 each with radial grooves 32 having micro-needles 35 fixedly secured therein, respectively. The micro-needles 35 are attached to the disk 31 with adhesive, and each of the circular disks 31 has a projection 33 from one side and a recess (not shown) formed in the other side in conformity with the projection 33 for close and tight assembly of the stack.

If the practitioner or the user holds the handle 10, brings the roller 30 into contact with the skin, and moves the micro-needle roller slowly, the roller 30 forms a plurality of micro-holes in the skin. The micro-holes in a surface layer of the skin formed thus serve to supply nutritive substance to the skin to regenerate the skin.

### DISCLOSURE OF INVENTION

### Technical Problem

In the related art micro-needle roller, the micro-needle is attached to the circular disk or formed as one unit with the circular disk at the time the circular disk is fabricated. The circular disk may be fabricated in sections like pizza pieces for assembly.

In the attachment type fabrication, if there is defect in attachment of the micro-needle, the micro-needle falls off the roller, or if excessive, sticks in the skin.

Moreover, the related art micro-needles are placed in the groove in one side of the circular disk and secured thereto with adhesive. Therefore, it has been very difficult to manage standard and quality of the product in making lengths of the needles projected beyond an outside circumference of the roller uniform and the like due to errors caused by machining errors both of the grooves and the needles, and errors caused by mounting of the needles to the grooves.

In the meantime, it is required to sterilize the micro-needle roller without fail for using the micro-needle roller, again. However, if the related art micro-needle roller is placed in a high temperature steam sterilizer or dipped in a sterilizing chemical, the circular disk of plastic deforms leading the needle attached to the circular disk with adhesive or formed as one unit with the circular disk too falls off the circular disk. Thus, as the needle is susceptible to fall off, the related art micro-needle roller can not, but be used as a disposable product, actually.

Furthermore, in the related art micro-needle roller, since it is required that the needles are placed in the radial grooves in the one side of the circular disk, it is required to increase the number of the grooves in the circular disk for increasing the number of the needles to be mounted to the roller. However, as shown in FIG. 2, in order to prevent the needle from falling off the circular disk easily, the groove extends from a center of the circular disk to an edge thereof extensively. Therefore, because the increase of the number of grooves in the circular disk is difficult, dense arrangement of the needles on the roller is not possible, resulting in poor effect of the practice due to failure in dense formation of the micro-holes in the skin.

Referring to FIG. 2, in the related art micro-needle roller, the circular disks are secured to the projections and recesses respectively, such that the cylindrical shape of stack of the circular disks are rotated as one unit. If the practitioner or the user rubs the skin with the roller while moving the roller back and forth, the needles projected from the surface of the roller keeps making holes in the surface of the skin repeatedly at a portion of the skin the needles have made holes, already. This is because all the needles rotate together with the roller.

The rotation of all the needles together with the roller can not but cause scratch at the skin in which the holes are made by the micro-needles positioned on an outer side circular disk when a curved portion of the skin is treated because a difference of rotation speeds between the outer side circular disk and an inner side circular disk can not be overcome. Particularly, if the rotation shaft stops suddenly in the middle of movement or the rotation is not smooth during use of the needle roller, the skin can be injured, or if excessive, a serious accident can happen, in which the skin tears.

Moreover, use of the related art micro-needle roller for user him(or her)self has restriction. That is, there is much inconvenience in practicing the micro-needle roller on his (or her) own body when the user him(or her)self holds the handle and practices the related art micro-needle roller because a rotation direction of the roller is fixed to the handle. For example, in a case the user practices his (or her) face or a portion of his (or her) body which is impossible to see with naked eyes for him (her) self, in general, a mirror is used. However, it is very difficult to make practice on an exact portion of the body the user desires with aid of the mirror because the handle having the rotation direction of the roller fixed thereto shades the body portion frequently in the practicing process.

Therefore, since the user is required to change a direction of a hand or the mirror in the practicing, use of the disk needle roller has been very inconvenient.

### Technical Solution

To achieve these objects and other advantages and in accordance with the purpose of the invention, as embodied and broadly described herein, a disk needle roller includes a handle having one pair of supporting bars extended therefrom, and a roller including a plurality of circular spacers placed on a rotation shaft having opposite ends supported on the supporting bars of the handle, a plurality of circular disks each between adjacent circular spacers so that the circular disks are rotatable around the rotation shaft independent from one another, a plurality of needles projected from an outside circumference of each of the disks in a radial direction, each with a tip formed sharply for forming micro-holes in skin of a human body.

In a front view of the disk, the needle has a right angular triangle, with perpendicular surface on an extension line from a center of the disk, and a sloped surface from a tip of the perpendicular surface to the disk.

In this case, one pair of adjacent needles have perpendicular surfaces and sloped surfaces arranged to face each other, respectively.

The disk needle roller further includes a head mounted turnable around a rotation shaft at an angle with respect to a length direction of the handle, and the roller is mounted rotatable around a supporting shaft arranged perpendicular to an extension line of the rotation shaft of the head.

One of the head and the handle has projections, and the other one of head and the handle has recesses in conformity with the projections so that the head is secured to the handle after the head is turned step by step at a desired angle.

One or more than one disks are mounted between adjacent spacers.

### ADVANTAGEOUS EFFECTS

The present invention has following advantageous effects. In the disk needle roller of the present invention, the needles are projected form a circumferential surface of the disk at one unit with the disk. Therefore, it is possible to prevent the needles from separating from disk caused during use or sterilization of the disk needle roller, enabling safe and many times of re-use of the disk needle roller without the problem of separation after sterilization of the disk needle roller. Moreover, since the needles are not required to assemble to the disk, quality control is very easy to provide an excellent effect of practice by making micro-holes in the skin at uniform depths.

The rotation of the disks independent from one another having the needles projected therefrom in the disk needle roller of the present invention permits to provide an excellent effect of practice since the disks form very dense micro-holes as the disks rotate independently when the disk needle roller is brought into contact with the skin and moves back and forth. Moreover, the independent rotation of the disks permits free practice on curved portions of the face, such as a lower jaw and both cheeks, and on heavily curved portion which is difficult to use, such as the bridge of a nose.

The head which rotatably supports the roller is rotatably mounted to the handle in the disk roller of the present invention. Therefore, because a rotation direction of the roller can be changed if a direction of the head is turned with respect to the handle, the user can make easy practice for him (her)self by using the disk needle roller of the present invention.

### BRIEF DESCRIPTION OF THE DRAWINGS

The accompanying drawings, which are included to provide further understanding of the disclosure and are incorporated in and constitute a part of this application, illustrate embodiments of the disclosure and together with the description serve to explain the principle of the disclosure.

In the drawings:

FIG. 1 illustrates a perspective view of a related art micro-needle roller;

FIG. 2 illustrates an exploded perspective view of the micro-needle roller in FIG. 1;

FIG. 3 illustrates a perspective view of a micro-needle roller in accordance with a preferred embodiment of the present invention;

FIG. 4 a plan view of a disk and needles applicable to the disk needle roller of the present invention;

FIG. 5 illustrates an exploded perspective view of a disk needle roller of the present invention;

FIG. 6 illustrates an exploded perspective view of a micro-needle roller in accordance with another preferred embodiment of the present invention;

FIG. 7 illustrates a perspective view of an assembly of the roller in FIG. 5;

FIG. 8 illustrates a section of the roller in FIG. 7;

FIG. 9 illustrates a perspective view of a micro-needle roller in accordance with a preferred embodiment of the present invention;

FIG. 10 illustrates an exploded perspective view of a fastening structure between a head and a handle in the disk needle roller of the present invention; and

FIG. 11 illustrates a perspective view of a disk needle roller of the present invention having a head turned with respect to a handle.

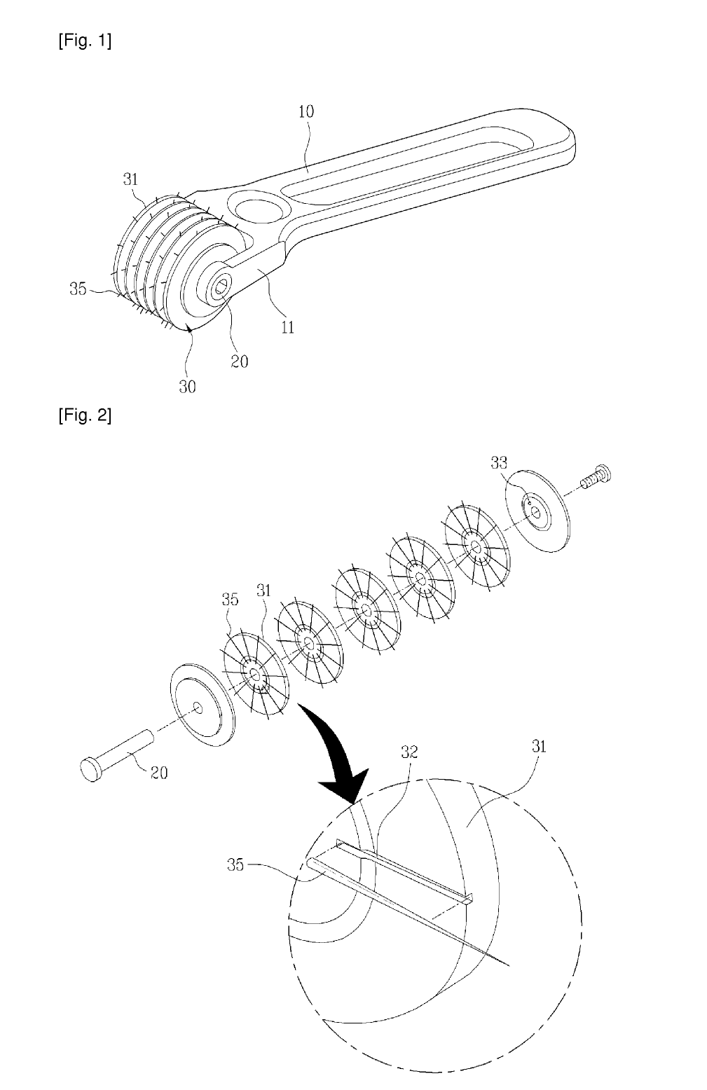

### BEST MODE FOR CARRYING OUT THE INVENTION

Reference will now be made in detail to the specific embodiments of the present invention, examples of which are illustrated in the accompanying drawings. Wherever possible, the same reference numbers will be used throughout the drawings to refer to the same or like parts.

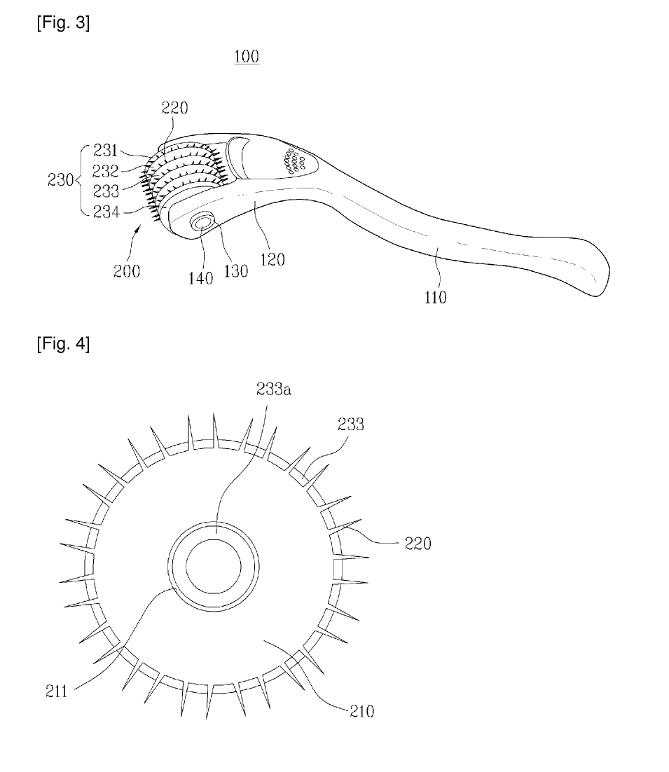

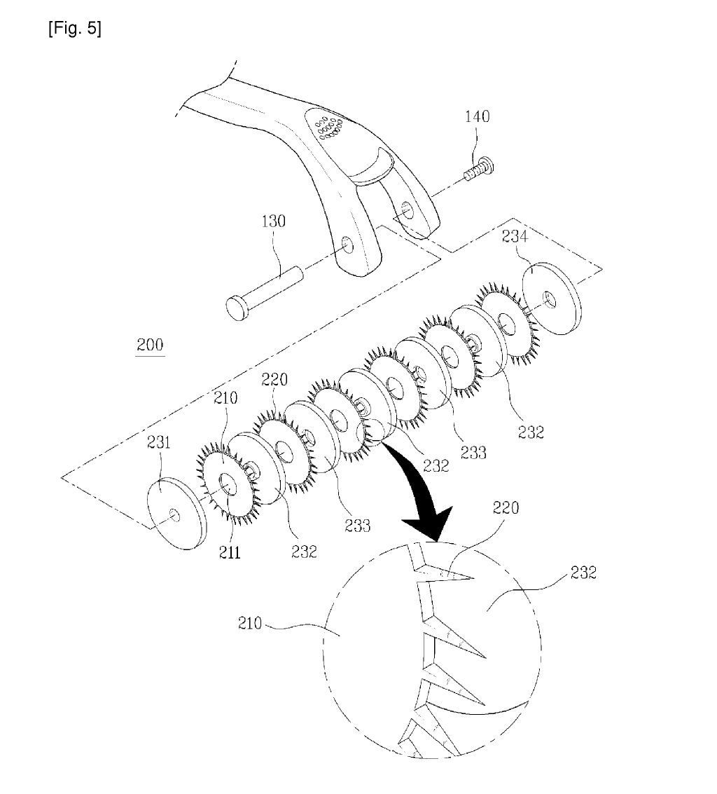

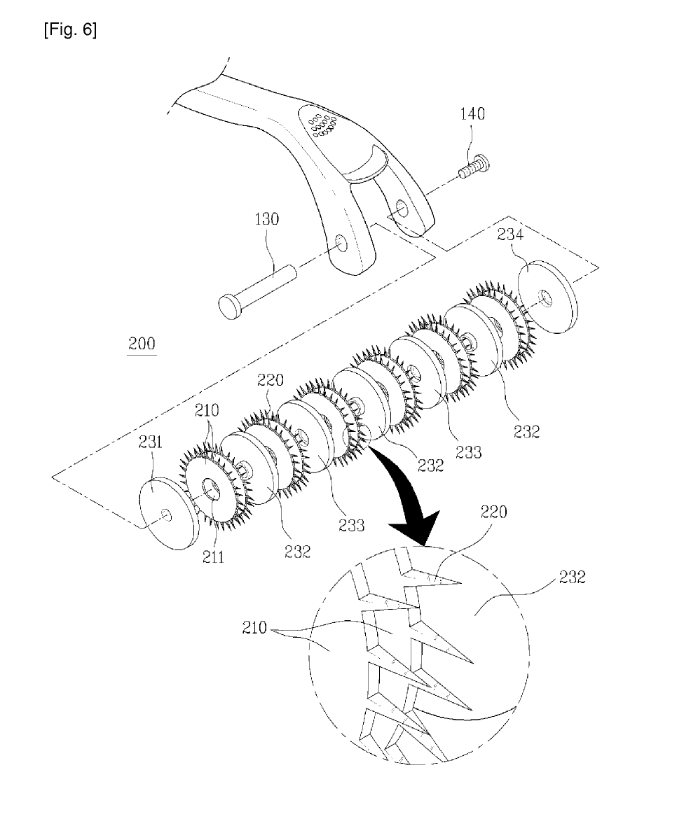

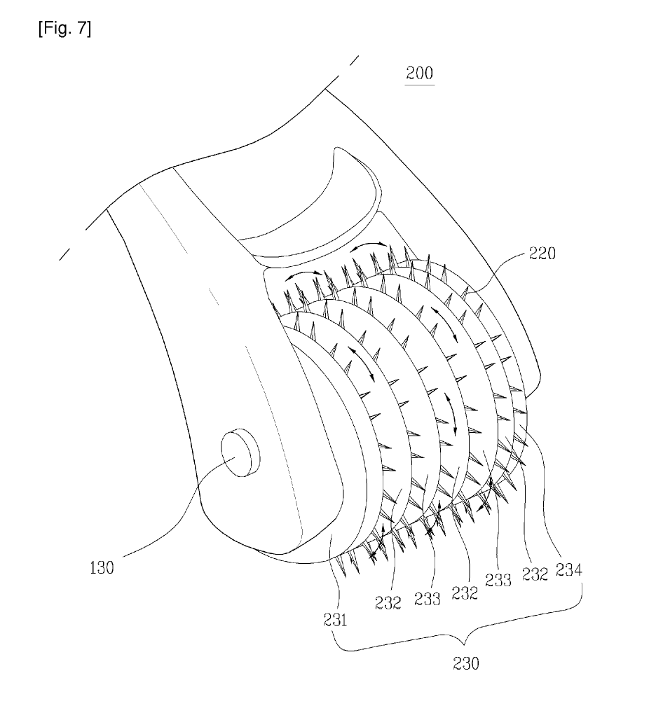

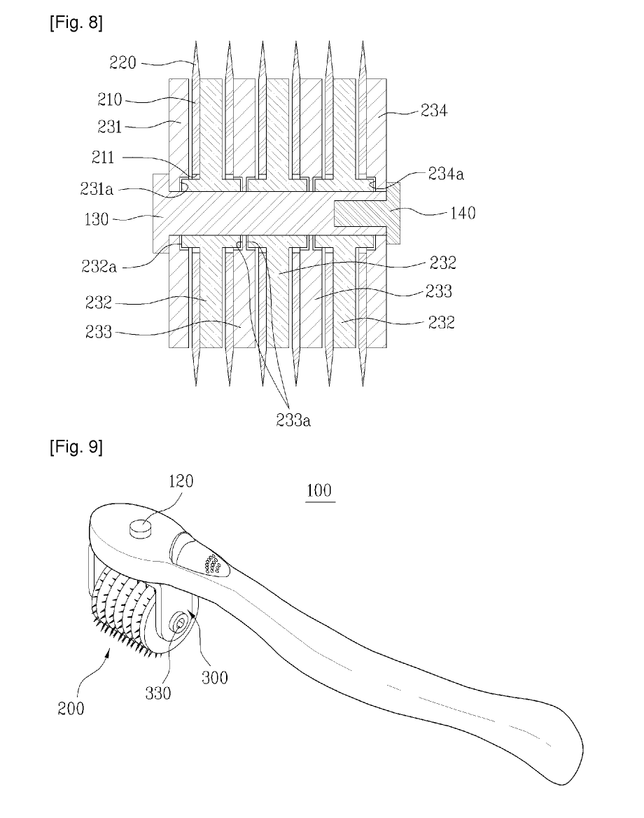

FIG. 3 illustrates a perspective view of a micro-needle roller in accordance with a preferred embodiment of the present invention, and FIG. 4 a plan view of a disk and needles applicable to the disk needle roller of the present invention. FIG. 5 and FIG. 6 illustrate an exploded perspective view of a disk needle roller of the present invention, and FIG. 7 illustrates a perspective view of an assembly of the roller in FIG. 5. And FIG. 8 is a cross sectional view of FIG. 7.

Referring to FIG. 3, the disk needle roller 100 of the present invention includes a handle 110 and a roller 200. The handle 110 is formed lengthy for easy holding by a practitioner or a user. The roller 200 is rotatably mounted to the handle 110. For this, the handle 110 has one end having one pair of supporting bars 120 extended forward in parallel, a roller 200 is arranged between the one pair of supporting bars 120, and a rotation shaft 130 is passed through both the roller 200 and the roller 200. According to this, the roller 200 is rotatable around the rotation shaft 130. At an end of the rotation shaft 130, there is a fastening bolt 140 fastened thereto.

Referring to FIG. 3, the roller 200 has a cylindrical exterior substantially, with a surface having a plurality of needles projected therefrom. The plurality of needles 220 projected from the surface of the roller 200 thus serve to make a plurality of microholes in skin when the user applies the disk needle roller 100 as the disk needle roller 100 is brought into contact with the skin.

Though the circular disks and the needles in the related art micro-needle roller are formed individually and bonded together with an adhesive, each of the disks 210 and the needles 220 in the micro-needle roller 100 of the present invention are formed as one unit. The roller 200 fabricated thus is shown in FIGS. 4 to 8 in detail, which will be described in more detail with reference to the drawings.

Referring to FIGS. 4 to 8, the roller 200 includes a stack of a plurality of disks 210 arranged parallel to one another, each with a plurality of needles 220 projected from a surface of the disk 210 in a radial direction.

Referring to FIGS. 4 and 5, the disk 210 has a circular disk with a predetermined thickness, with a pass through hole 211 at a center for pass of a rotation shaft 130. As shown in FIG. 5, the plurality of disks 210 are stacked inserted on the rotation shaft 130 in an orderly fashion arranged parallel to one another.

Referring to FIGS. 4 and 5, there can be a circular spacer 230 arranged between each of adjacent disks 210 and on an outer side of each of foremost and aftermost disks 210.

The spacer 232 or 233 arranged between each of adjacent disks 210 serves to make the adjacent disks 210 spaced away from each other, and the spacer 231 or 234 arranged on an outer side of each of foremost and aftermost disks 210 serves to protect the foremost and aftermost disks from an outside.

The spacer 230 has a diameter greater than the disk 210 so that a circumferential surface of the disk 210 is not exposed to an outside of the disk needle roller for the needles 220 to appear projected to the outside of the disk needle roller between adjacent spacers 230.

It is preferable that the spacer 230 includes a female spacer 231, 233, and 234 having a central recess 231a, 233a, or 234a in each of at least either front or back side of the circular spacer body, and a male spacer 232 having a central projection 232a from each of front and back sides of the circular spacer body to be placed in the recess 231a, 233a or 234a of the female spacer 231, 233, or 234. In this case, the female spacer 231, 233 or 234 and the male spacer 232 are assembled as the recess 231a, 233a or 234a and the projection 232a are placed in together, and the disk 210 is assembled in a state the pass through hole 211 is placed on the projection 232a of the male spacer 232.

Accordingly, the spacer 230 and the disk 210 may rotate independent from one another.

In the meantime, referring to FIG. 6, a plurality of disks 210 may be mounted between adjacent spacers 230. The mounting of the plurality of disks 210 between adjacent spacers 230, arranging the needles 220 closer, enables convenient use of the disk needle roller in making holes at portions with local wrinkles, such as under the eyes and forehead, sensitive portions, and narrow grooved portions, such as nose.

Therefore, the number of disks 210 arranged between the disks 210 can be adjusted freely suitable for portion the needle roller is applied thereto. For example, the spacer 230 may be formed of plastic.

Referring to FIG. 4, the plurality of needles 220 are projected from the circumferential surface of the disk 210 in a radial direction. The needles are formed as a unit with the disk 210, arranged closely along a circumferential direction of the disk 210. Though the disk 210 and the needle 220 can be formed of a variety of materials, it is preferable that the disk 210 and the needle 220 are formed of a metal, such as stainless steel that shows no deformation even in sterilization with high temperature and chemicals. If the disk 210 and the needle 220 are formed of a metal, such as stainless steel, a sense of weight on the head side, i.e., the roller 200 side of the product provides a sense of convenience of use of the disk needle roller, and is convenient since needles 220 are brought into close contact with, and inserted in, the skin naturally even if the handle is not pressed down with great force during use of the disk needle roller.

In the disk needle roller 100 of the present invention, since the needle 220 is formed as one unit with the disk 210, the needle 220 has no possibility of separation from the disk 210. Therefore, since there is no possibility of the needle 220 to fall off the disk 210, or stick in the skin, in using the disk needle roller 100 at all, the disk needle roller 100 can be used safely. Moreover, since the needle 220 has no possibility of separation from the disk 210 in use or in sterilization, the disk needle roller 100 can be re-used after sterilizing the disk needle roller 100 for many times.

Different from the related art, the disk needle roller of the present invention requires no formation of the grooves for attaching the needles thereto, or attachment of the needles to the circular disk with an adhesive, at all. Eventually, the disk needle roller of the present invention has a simple structure and easy to fabricate. Moreover, the disk needle roller of the present invention can have easy standard and quality management, enabling to make micro-holes in the skin at uniform depths, thereby permitting an effective practice.

Since the needles are inserted down close to the center of the circular disk in the related art micro-needle roller, there has been a limitation in close arrangement of the needles on the outside circumferential surface of the roller in view of a structure of the disk needle roller. Opposite to this, since the needles and the disk 210 are formed as one unit in the present invention, the needles 220 can be arranged closely along the circumferential surface of the disk 210 having a comparably long length. Therefore, the disk needle roller of the present invention permits an arrangement of the needles 220 closer than the related art disk needle roller, enabling dense formation of the microholes in the skin to provide an excellent practicing effect.

Referring to FIG. 5, in the disk needle roller 100 of the present invention, there is no member, such as a key, between the rotation shaft 130 and the disks 210 for rotating the rotation shaft 130 and the disks 210 as one body. Moreover, different from the related art disk needle roller, adjacent disks 210 are not fastened with a projection and a recess. Accordingly, as shown in FIG. 8, the disks 210 are rotatable around the rotation shaft 130 independent from one another.

When the disk needle roller 100 is used, the practitioner or the user brings the roller 200 into contact with the skin and moves the roller 200 back and forth, when each of the disks 210 in the roller 200 rotates around the rotation shaft 130 independent from each other as shown in FIG. 8. Since such a irregular rotation of the disks 210, letting the needles 220 of the disks 210 to make holes at other portions of the skin always even if the roller 200 moves back and forth, side effects of damage to the skin and scars formation caused by repetitive hole making at the same point can be prevented, and denser micro-holes than the related art disk needle roller can be made, effectively.

The independent rotation of the spacers 230 and the disks 210 enables to prevent the needles 230 from damaging the skin despite of sudden stopping motion of the disk needle roller 100.

Since the disk needle 210 is assembled in a state the pass through hole 211 is placed on the projection 232a at the center of the male spacer 232, the disk needle 210 can make smoother and stable rotation.

Referring to FIG. 4, in the disk needle roller 100 of the present invention, each needle 220 has a right triangular longitudinal section. This is for making a tip thereof sharper. Along with this, by rounding sharp corners of the needle are rounded later, the tip of the needle may be made sharper.

That is, when the needle 220 is seen from a front of the disk 210, the needle 220 has a right angular shape having a perpendicular surface on an extension line passing through the center of the disk 210, and a sloped surface starting from the tip of the perpendicular surface to the disk 210.

Referring to FIG. 4, one pair of adjacent needles 220 and 220 have the perpendicular surfaces and the sloped surfaces arranged to face each other, respectively. Above arrangement of the needles 220 makes distances between the needles 220 different from one another slightly. As a result, when the roller 200 moves back and forth, since the disks 210 rotate independent from one another irregularly, for the needles to make the micro-holes in the skin, not coarsely with a fixed direction, but very densely without fixed direction, the effect of the practice can be enhanced further.

If the plurality of micro-holes are formed in the skin with the disk needle roller 100 of the present invention, the plurality of micro-holes stimulates the skin to enhance a natural healing capability of the skin, enabling to obtain excellent effects not only in skin beauty, but also in healing of skin disease, or a scar such as burn. Especially, if a medicine or cosmetic which helps regeneration of the skin or nourishing the skin is applied, the skin regeneration and nourishing effect can be maximized.

In the meantime, referring to FIGS. 9 to 11, it is preferable that the disk needle roller 100 of the present invention further includes a head 300 mounted rotatable around a rotation shaft 120 at a desired angle with respect to a length direction of the handle 110, so that the roller 200 rotates around a supporting shaft 330 arranged perpendicular to an extension line of the rotation shaft 120 of the head 300.

The handle 110 has one end with a shaft hole 111 for passing of the rotation shaft 120, wherein the head 300 is rotatably mounted to the handle 110 with the rotation shaft 120 passed through the shaft hole 111.

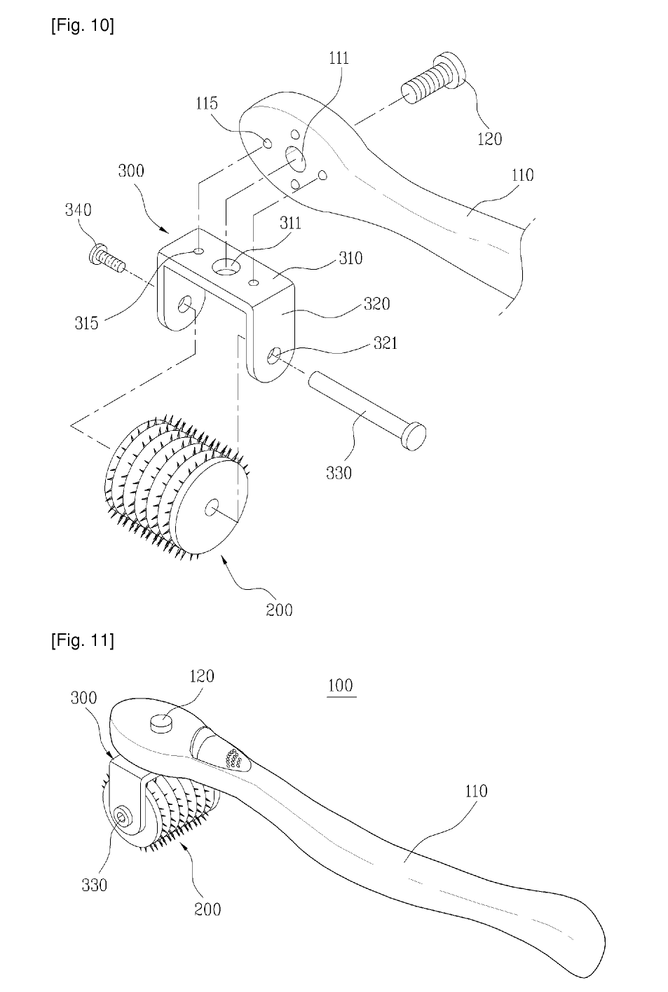

Though the rotation shaft 120 can be arranged in a length direction of the handle 110, it is preferable that the rotation shaft 120 is arranged at an angle with respect to the length direction of the handle 110, for an example, perpendicular to the length direction of the handle 110 as shown in FIG. 10, substantially. If the rotation shaft 120 crosses the handle 110 substantially, the head 300 is mounted to a side of one end of the handle 110. The head 300 mounted thus is rotatable around the rotation shaft 120.

Referring to FIG. 9, the head 300 includes a supporting plate 310 rotatably mounted to the handle 110 with the rotation shaft 120, and one pair of extensions 320 from opposite ends of the supporting plate 310 parallel to each other.

The supporting plate 310 is parallel to the handle 110 substantially, with a hole 311 at a center for placing the rotation shaft 120 therein. Each of the extensions 320 has a supporting shaft hole 321 formed therein, for placing the supporting shaft 330 therein which rotatably supports the roller 200.

The head 300 formed thus can be turned around the rotation shaft 120 at a desired angle with respect to the handle 110 step by step and fixes to the desired angle. In more detail, as shown in FIG. 10, the handle 110 has projections 115, and the supporting plate 310 of the head 300 has recesses 315 in conformity with the projections 115. Of course, the projections 115 and the recesses 315 may be formed at opposite positions. The projections 115 are formed at regular angle intervals, for example, at 90 degree intervals along a rotation direction of the head 300.

Accordingly, the head 300 can be secured at every 90 degree rotation by the projection 115 and the recess 315, firmly. In the meantime, if necessary, by changing locations and the number of the projections 115, the stepwise turning angles of the head 300 can be adjusted to be below the 90 degrees.

The roller 200 is substantially cylindrical and arranged between the extensions 320 of the head 300. The roller 200 arranged thus is mounted to the head 300 to rotate around the supporting shaft 330 passed through the extensions 320.

Thus, in the disk needle roller 100 of the present invention, the head 300 turns around the rotation shaft 120 at an angle with respect to the length direction of the handle 110, and the roller 200 rotates around the supporting shaft 330 perpendicular to an extension line (not shown) of the rotation shaft 120, substantially.

Then, the user can select a direction convenient to use to the user, turns the head 300 to a position as shown in FIG. 9 or 11, and practices easily and conveniently for him (her)self. For reference, when the head 300 is arranged in a same direction with the handle 110 as shown in FIG. 9, the user may practice by moving the handle 110 in left/right directions, and when the head 300 is arranged in a direction as shown in FIG. 11, the user may practice by moving the handle 110 in up and down directions. Thus, the user can use the disk needle roller after the user selects a convenient direction and turns the head 300 to the direction.

It will be apparent to those skilled in the art that various modifications and variations can be made in the present invention without departing from the spirit or scope of the invention.

Thus, it is intended that the present invention cover the modifications and variations of this invention provided they come within the scope of the appended claims and their equivalents.

### INDUSTRIAL APPLICABILITY

The present invention has an industrial applicability. “Refer to Advantageous effects”

1. A disk needle roller comprising:
a handle having one pair of supporting bars extended therefrom; and
a roller including;
a plurality of circular spacers placed on a rotation
shaft having opposite ends supported on the supporting bars of the handle,
a plurality of circular disks each between adjacent circular spacers so that the
circular disks are rotatable around the rotation shaft independent from one another,
a plurality of needles projected from an outside circumference of each of the disks in a radial direction, each with a tip formed sharply for forming microholes in skin of a human body.

2. The disk needle roller as claimed in claim 1, wherein, in a front view of the disk, the needle has a right angular triangle, with perpendicular surface on an extension line from a center of the disk, and a sloped surface from a tip of the perpendicular surface to the disk.

3. The disk needle roller as claimed in claim 2, wherein one pair of adjacent needles has perpendicular surfaces and sloped surfaces arranged to face each other, respectively.

4. The disk needle roller as claimed in claim 1, further comprising a head mounted turnable around a rotation shaft at an angle with respect to a length direction of the handle, and the roller is mounted rotatable around a supporting shaft arranged perpendicular to an extension line of the rotation shaft of the head.

5. The disk needle roller as claimed in claim 4, wherein one of the head and the handle has projections, and the other one of head and the handle has recesses in conformity with the projections so that the head is secured to the handle after the head is turned step by step at a desired angle.

6. The disk needle roller as claimed in claim 1, wherein one or more than one disks is mounted between adjacent spacers.
